(19) United States
(12) Patent Application Publication
(10) Pub. No.: US 2025/0057745 A1
(43) Pub. Date: Feb. 20, 2025

(54) USE OF A PARTICULAR CARBOXYLIC ACID OR SALTS THEREOF FOR REPAIRING AND/OR PREVENTING BREAKAGE OF KERATIN FIBRES

(71) Applicant: L'Oreal, Paris (FR)

(72) Inventor: Andrew Greaves, Paris (FR)

(73) Assignee: L'Oreal, Paris (FR)

(21) Appl. No.: 18/720,187

(22) PCT Filed: Dec. 14, 2022
(86) PCT No.: PCT/EP2022/085924

§ 371 Date: Jun. 14, 2024

(30) Foreign Application Priority Data
Dec. 15, 2021 (FR) FR2113586

(51) Int. Cl. A61K 8/44; A61Q 5/00

(52) U.S. Cl. CPC A61K 8/44; A61Q 5/00; A61Q 5/002

(57) ABSTRACT

The present invention relates to the use of a particular carboxylic acid or of its salts for repairing and/or preventing the breakage of keratin fibres, more particularly human keratin fibres such as the hair. The invention also relates to a cosmetic composition comprising at least one particular carboxylic acid or its salts and at least one anionic surfactant. The invention also pertains to a method for cosmetic treatment of keratin fibres, more particularly human keratin fibres such as the hair, using said particular carboxylic acid or its salts or said cosmetic composition.

The present invention relates to the use of a particular carboxylic acid or of its salts for repairing and/or preventing the breakage of keratin fibres, more particularly human keratin fibres such as the hair.

The invention also relates to a cosmetic composition comprising at least one particular carboxylic acid or its salts and at least one anionic surfactant.

The invention also pertains to a method for cosmetic treatment of keratin fibres, more particularly human keratin fibres such as the hair, using said particular carboxylic acid or its salts or said cosmetic composition.

The hair may be damaged and made brittle by external atmospheric agents such as pollution and bad weather, and also by mechanical or chemical treatments, or certain routines, such as brushing, combing, dyeing, bleaching, permanent-waving, straightening and/or repeated washing. The hair may then end up damaged and may in the long run become dry, coarse, brittle, dull, split and/or limp.

To overcome these disadvantages, then, it is common practice to employ haircare compositions intended for conditioning the hair by giving it satisfactory cosmetic properties, notably smoothness, sheen, a soft feel, suppleness and lightness, and also good disentangling properties leading to easy combing and good manageability of the hair, which is thus easier to style and holds its shape. However, the conditioning effect obtained via these haircare compositions fades out rapidly over time and does not allow the hair cortex to be strengthened.

As an example, shampoos are typically used to cleanse the hair, imparting to it satisfactory cosmetic properties, but these compositions do not improve the quality of the fibre. In particular they do not allow the density of linkages between the proteins present in the cortex of the fibre to be improved, so as to repair the fibre or reduce its breakage, during combing or disentangling, for example.

There is therefore still a genuine need to find a means of treating keratin fibres, more particularly human keratin fibres such as the hair, preferably those which have been damaged and/or sensitized, that is capable of conserving or even improving the quality of said fibres, by increasing in particular the density of linkages between the proteins present in the cortex of these fibres, so allowing them to be repaired and/or their breakage to be reduced, as during combing or disentangling, for example.

It has now been found that the use of a particular polycarboxylic acid, optical isomers thereof, geometric isomers thereof, salts thereof and/or solvates thereof allows the objectives set out above to be met, and particularly allows keratin fibres to be repaired and/or their breakage to be reduced, by increasing in particular the bonding density between the proteins present in the cortex of said fibres.

A subject of the present invention is thus the use of one or more carboxylic acids of formula (I) below, or optical isomers thereof, geometric isomers thereof, salts thereof, solvates thereof and mixtures thereof:

R₁—N—(CH(R₂)COOH)₂ (I)

in which:
R₁ represents a hydrogen atom or a —CH(COOH)—(CH₂)₂—COOH, —CH₂CH₂OH, —CH(CH₃)COOH, —(CH₂)₂N(COR₃)—CH₂—COOH or —CH(COOH)—CH₂—COOH group; and
R₂ represents a CH₂COOH group when R₁ represents a hydrogen atom or R₂ represents a hydrogen atom when R₁ is other than a hydrogen atom; and
R₃ represents a linear or branched alkyl group comprising from 1 to 4 carbon atoms or cyclic alkyl group comprising from 3 to 30 carbon atoms,
for repairing and/or preventing the breakage of keratin fibres, more particularly human keratin fibres such as the hair.

The use of carboxylic acids of formula (I), or optical isomers thereof, geometric isomers thereof, salts thereof, solvates thereof and mixtures thereof, according to the invention makes it possible to repair and/or prevent the breakage of keratin fibres, preferably those which have been damaged and/or sensitized. This is because the use of these carboxylic acids of formula (I) increases the bonding density between the proteins present in the cortex of said fibres. These fibres are then more resistant to external stresses, especially to combing and/or disentangling.

These properties are also persistent with respect to several shampooing operations. The keratin fibres thus treated are repaired and/or protected, in a long-lasting manner, with respect to external agents.

These keratin fibres also exhibit enhanced cosmetic properties in terms especially of sheen, sleekness and manageability. Their feel is notably softer and more natural.

The keratin fibres repaired and/or protected according to the present invention are preferably damaged and/or sensitized keratin fibres, more particularly damaged and/or sensitized human keratin fibres such as damaged and/or sensitized hair.

“Damaged fibres” in the sense of the present invention are dry, coarse, brittle, split and/or limp fibres.

“Sensitized fibres” in the sense of the present invention are bleached, artificially coloured, straightened and/or permanent-waved fibres.

Another subject of the present invention is a cosmetic composition comprising:
one or more carboxylic acids of formula (I), or optical isomers thereof, geometric isomers thereof, salts thereof, solvates thereof and mixtures thereof, as defined above, which are present in a total amount ranging from 2% to 25% by weight, relative to the total weight of the composition, and
one or more anionic surfactants.

The cosmetic composition of the invention provides in particular a good level of detergence to the keratin fibres, while increasing the bonding density between the proteins present in the cortex of said fibres.

Another subject of the present invention relates to a method for cosmetic treatment of keratin fibres, more particularly human keratin fibres such as the hair, comprising applying to said keratin fibres one or more carboxylic acids of formula (I), or optical isomers thereof, geometric isomers thereof, salts thereof, solvates thereof and mixtures thereof, or a cosmetic composition as defined above.

Other subjects, features, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

### Use

The present invention pertains to the use of one or more carboxylic acids of formula (I), as defined above, or optical isomers thereof, geometric isomers thereof, salts thereof, solvates thereof and mixtures thereof, for repairing and/or preventing the breakage of keratin fibres, more particularly human keratin fibres such as the hair.

More specifically, the carboxylic acids of formula (I) correspond to:
compounds comprising four carboxylic acid functions, when R₁ represents a hydrogen atom and R₂ represents a —CH₂—COOH group, or when R₁ represents the —CH(COOH)—(CH₂)₂—COOH group and R₂ represents a hydrogen atom or when R₁ represents the —CH(COOH)—CH₂—COOH group and R₂ represents a hydrogen atom;
compounds comprising three carboxylic acid functions, when R₁ represents the —CH(CH₃)—COOH group and R₂ represents a hydrogen atom, or when R₁ represents a —(CH₂)₂—N(COR₃)—CH₂—COOH group and R₂ represents a hydrogen atom; and to
compounds comprising two carboxylic acid functions, when R₁ represents the —CH₂CH₂OH group and R₂ represents a hydrogen atom.

The carboxylic acids of formula (I) may take the form of pure enantiomers, preferably in L configuration, or the form of mixtures, especially of racemic mixtures.

The salts of the one or more carboxylic acids of formula (I) are preferably selected from alkali metal salts, alkaline earth metal salts, transition metal salts, organic amine salts, ammonium salts and mixtures thereof.

Examples of alkali metal salts include especially sodium (Na*) and potassium (K⁺) salts, whereas examples of alkaline earth metal salts include especially calcium (Ca²⁺) and magnesium (Mg²⁺) salts.

For the purposes of the present invention, a “transition metal” is a metal comprising an incomplete d subshell, and more particularly in the II oxidation state, such as cobalt (Co²⁺), iron (Fe²⁺), manganese (Mn²⁺), zinc (Zn²⁺) and copper (Cu²⁺).

As regards the organic amine salts, mention may be made of the salts of primary, secondary or tertiary amines, or alternatively of alkanolamines. Said amines exhibit one or more identical or non-identical radicals, of linear or branched C₁ to C₂₀ alkyl type, optionally comprising a heteroatom such as oxygen.

Preferably, said one or more carboxylic acids of formula (I) used in the context of the present invention are selected from methylglycinediacetic acid, N-lauroylethylenediamine-N,N′,N′-triacetic acid, N,N-dicarboxymethylglutamic acid, iminodisuccinic acid, N,N-bis(carboxymethyl)aspartic acid, alkali metal salts thereof, alkaline earth metal salts thereof, transition metal salts thereof, organic amine salts thereof, ammonium salts thereof, optical isomers thereof, geometric isomers thereof, solvates thereof and mixtures thereof, and more preferably from N,N-dicarboxymethylglutamic acid, N,N-bis(carboxymethyl)aspartic acid, alkali metal salts thereof, alkaline earth metal salts thereof, transition metal salts thereof, organic amine salts thereof, ammonium salts thereof, optical isomers thereof, geometric isomers thereof, solvates thereof and mixtures thereof.

Better still, the one or more carboxylic acids of formula (I) are selected from N,N-dicarboxymethyl-L-glutamic acid (GLDA), N,N-bis(carboxymethyl)-L-aspartic acid, alkali metal salts thereof, alkaline earth metal salts thereof, transition metal salts thereof, organic amine salts thereof, ammonium salts thereof, solvates thereof and mixtures thereof.

Preferably, the carboxylic acid of formula (I) used according to the present invention is N,N-dicarboxymethyl-L-glutamic acid (GLDA) and/or tetrasodium N,N-bis(carboxymethyl)-L-glutamate.

N,N-Dicarboxymethyl-L-glutamic acid, tetrasodium N,N-bis(carboxymethyl)-L-glutamate and N,N-bis(carboxymethyl)aspartic acid are respectively represented by formulae (II), (III) and (IV) below:

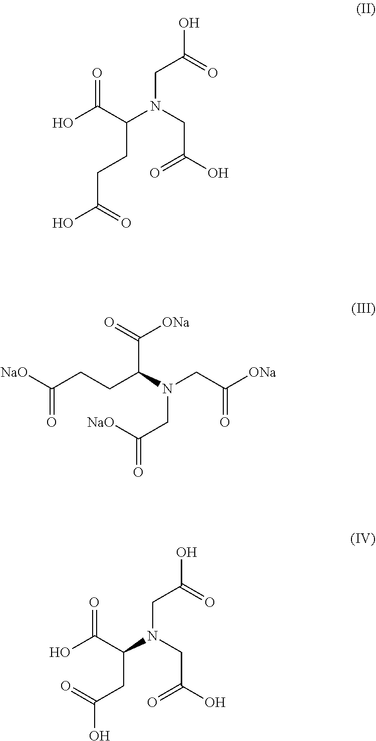

According to one particular embodiment of the invention, the one or more carboxylic acids of formula (I), as defined above, are advantageously present in a composition, preferably an aqueous composition. Another subject of the present invention thus pertains to the use of a composition comprising one or more carboxylic acids of formula (I) as defined above for repairing and/or preventing the breakage of keratin fibres, more particularly human keratin fibres such as the hair.

According to this embodiment, the total amount of the one or more carboxylic acids of formula (I) as defined above that are present in the composition ranges preferably from 0.01% to 25% by weight, more preferentially from 1% to 25% by weight, and better still from 2% to 20% by weight, relative to the total weight of the composition.

According to a first embodiment, the one or more carboxylic acids of formula (I), optical isomers thereof, geometric isomers thereof, salts thereof, solvates thereof and mixtures thereof, as defined above, are used for repairing and/or preventing the breakage of keratin fibres, more particularly human keratin fibres such as the hair, which have been damaged.

According to another embodiment, the one or more carboxylic acids of formula (I), optical isomers thereof, geometric isomers thereof, salts thereof, solvates thereof and mixtures thereof, as defined above, are used for repairing and/or preventing the breakage of keratin fibres, more particularly human keratin fibres such as the hair, which have been sensitized.

### The Composition

Another subject of the present invention is a cosmetic composition, preferably aqueous, comprising:
one or more carboxylic acids of formula (I), or optical isomers thereof, geometric isomers thereof, salts thereof, solvates thereof and mixtures thereof, as defined above, or salts thereof, which are present in a total amount ranging from 2% to 25% by weight, relative to the total weight of the composition, and
one or more anionic surfactants.

The total amount of the one or more carboxylic acids of formula (I) as defined above that are present in the cosmetic composition according to the invention ranges from 2% to 25% by weight, and more preferentially from 2% to 20% by weight, relative to the total weight of the composition.

The term “anionic surfactant” means a surfactant including, as ionic or ionizable groups, only anionic groups.

In the present description, a species is termed as being “anionic” when it bears at least one permanent negative charge or when it can be ionized to a negatively charged species, under the conditions of use of the composition of the invention (for example the medium or the pH) and not comprising any cationic charge.

The one or more anionic surfactants may be selected from sulfate, sulfonate and/or carboxylic (or carboxylate) surfactants. Needless to say, a mixture of these surfactants may be used.

It is understood in the present description that:
the carboxylate-type anionic surfactants comprise at least one carboxylic or carboxylate function (—COOH or —COO⁻) and may optionally also comprise one or more sulfate and/or sulfonate functions;
the sulfonate-type anionic surfactants comprise at least one sulfonate function (—SO₃H or —SO₃) and may optionally also comprise one or more sulfate functions, but do not comprise any carboxylate functions; and
the sulfate-type anionic surfactants comprise at least one sulfate function but do not comprise any carboxylate or sulfonate functions.

The carboxylate-type anionic surfactants that may be used in the composition of the invention thus include at least one carboxylic or carboxylate function (—COOH or —COO⁻).

They may be chosen from the following compounds: acyl glycinates, acyl lactylates, acyl sarcosinates, acyl glutamates; alkyl-D-galactosideuronic acids, alkyl ether carboxylic acids, alkyl(C₆-C₃₀ aryl) ether carboxylic acids, alkylamido ether carboxylic acids; and also the salts of these compounds; and mixtures thereof; the alkyl and/or acyl groups of these compounds including from 6 to 30 carbon atoms, preferably from 8 to 26, and more preferentially from 10 to 22 carbon atoms; the aryl group preferably denoting a phenyl or benzyl group;
these compounds possibly being polyoxyalkylenated, notably polyoxyethylenated, and then preferably including from 1 to 50 ethylene oxide units and better still from 2 to 10 ethylene oxide units.

Use may also be made of C₆-C₃₀ alkyl monoesters of polyglycoside-polycarboxylic acids such as C₆-C₃₀ alkyl polyglycoside-citrates, C₆-C₃₀ alkyl polyglycoside-tartrates and C₆-C₃₀ alkyl polyglycoside-sulfosuccinates, and salts thereof.

Preferentially, the carboxylate anionic surfactants are chosen, alone or as a mixture, from:
acyl glutamates, notably C₆-C₃₀ or even C₈-C₂₆ acyl glutamates, such as stearoyl glutamates, and in particular disodium stearoyl glutamate;
acyl sarcosinates, notably C₆-C₃₀ or even C₈-C₂₆ acyl sarcosinates, such as palmitoyl sarcosinates, and in particular sodium palmitoyl sarcosinate;
acyl lactylates, notably C₆-C₃₀ or even C₈-C₂₆ acyl lactylates, such as behenoyl lactylates, and in particular sodium behenoyl lactylate;
C₆-C₃₀ and notably C₈-C₂₆ acyl glycinates;
(C₆-C₃₀)alkyl ether carboxylates, and notably (C₈-C₂₆)alkyl ether carboxylates;
polyoxyalkylenated (C₆-C₃₀)alkyl(amido) ether carboxylic acids, in particular those including from 2 to 50 ethylene oxide groups;
in particular in the form of alkali metal or alkaline-earth metal, ammonium or amino alcohol salts.

Among the above carboxylate-type surfactants, mention may be made most particularly of surfactants of fatty acid type, notably of C₆-C₃₀. These surfactants are preferably chosen from the compounds of formula (a) below:

R—C(O)—OX (a)

with
X denoting a hydrogen atom, an ammonium ion, an ion resulting from an alkali metal or alkaline earth metal or an ion resulting from an organic amine, preferably a hydrogen atom, and
R denoting a linear or branched, saturated or unsaturated alkyl group of 5 to 29 carbon atoms.

Preferably, R denotes a linear or branched, saturated or unsaturated alkyl group of 7 to 23 carbon atoms, preferably of 11 to 21 carbon atoms.

Among the above carboxylate-type surfactants, mention may be made most particularly of surfactants of sarcosinate type, notably selected from the (C₆-C₃₀)acyl sarcosinates of formula (V) below:

R—C(O)—N(CH₃)—CH₂—C(O)—OX (V)

with
X denoting a hydrogen atom, an ammonium ion, an ion resulting from an alkali metal or alkaline earth metal or an ion resulting from an organic amine, preferably a hydrogen atom, and
R denoting a linear or branched alkyl group of 5 to 29 carbon atoms.

Preferably, R denotes a linear or branched alkyl group of 8 to 24 carbon atoms, preferably of 12 to 20 carbon atoms.

Among the (C₆-C₃₀)acyl sarcosinates of formula (V) that can be used in the present composition, mention may be made of palmitoyl sarcosinates, stearoyl sarcosinates, myristoyl sarcosinates, lauroyl sarcosinates and cocoyl sarcosinates, in acid form or in salified form.

Among the above carboxylate-type surfactants, mention may also be made of polyoxyalkylenated alkyl(amido) ether carboxylic acids and salts thereof, in particular those including from 2 to 50 alkylene oxide and in particular ethylene oxide groups, such as the compounds sold by the company Kao under the Akypo names.

The polyoxyalkylenated alkyl(amido) ether carboxylic acids that may be used are preferably selected from those of formula (VI):

R₁—(OC₂H₄)ₙ—OCH₂COOA (VI)

in which:
R₁ represents a linear or branched C₆-C₂₄ alkyl or alkenyl radical, a (C₈-C₉)alkylphenyl radical, a radical R₂CONH—CH₂—CH₂— with R₂ denoting a linear or branched C₉-C₂₁ alkyl or alkenyl radical;
preferably, R₁ is a C₈-C₂₀ and preferably C₈-C₁₈ alkyl radical, and aryl preferably denotes phenyl,
n is an integer or decimal number (mean value) ranging from 2 to 24 and preferably from 2 to 10,
A denotes H, ammonium, Na, K, Li, Mg or a monoethanolamine or triethanolamine residue.

Use may also be made of mixtures of compounds of formula (VI), more particularly mixtures of compounds having different groups R1.

The polyoxyalkylenated alkyl(amido) ether carboxylic acids that are particularly preferred are those of formula (VI) in which:
R₁ denotes a C₁₂-C₁₄ alkyl, cocoyl, oleyl, nonylphenyl or octylphenyl radical,
A denotes a hydrogen or sodium atom, and
n ranges from 2 to 20, preferably from 2 to 10.

Even more preferentially, use is made of the compounds of formula (VI) in which R₁ denotes a C₁₂ alkyl radical, A denotes a hydrogen or sodium atom and n ranges from 2 to 10.

The sulfonate-type anionic surfactants that may be used in the composition of the invention include at least one sulfonate function (—SO₃H or —SO₃⁻).

They may be selected from the following compounds: alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, N-acyl taurates, acyl isethionates; alkyl sulfolaurates; and also the salts of these compounds;
the alkyl groups of these compounds including from 6 to 30 carbon atoms, notably from 12 to 28, even better still from 14 to 24 or even from 16 to 22 carbon atoms; the aryl group preferably denoting a phenyl or benzyl group;
these compounds possibly being polyoxyalkylenated, notably polyoxyethylenated, and then preferably including from 1 to 50 ethylene oxide units and better still from 2 to 10 ethylene oxide units.

Preferentially, the sulfonate anionic surfactants are selected, alone or as a mixture, from:
C₆-C₂₄ and notably C₁₂-C₂₀ alkyl sulfosuccinates, notably lauryl sulfosuccinates;
C₆-C₂₄ and notably C₁₂-C₂₀ alkyl ether sulfosuccinates;
C₆-C₂₄ and notably C₁₂-C₂₀ N-acyl taurates;
(C₆-C₂₄)acyl isethionates, preferably (C₁₂-C₁₈)acyl isethionates; in particular in the form of alkali metal or alkaline-earth metal, ammonium or amino alcohol salts.

Preferably, the one or more anionic surfactants are selected from sulfate-type anionic surfactants and mixtures thereof.

For the purposes of the present invention, the term “sulfate-type anionic surfactant” means an anionic surfactant including one or more sulfate functions (—OSO₃H or —OSO₃⁻).

Such surfactants may advantageously be selected from alkyl sulfates, alkyl ether sulfates, alkylamido sulfates, alkylamido ether sulfates, alkylaryl polyether sulfates, monoglyceride sulfates; and also salts thereof and mixtures thereof; the alkyl groups of these compounds notably including from 6 to 30 carbon atoms, preferably from 8 to 26, and more preferentially from 10 to 22 carbon atoms; the aryl group preferably denoting a phenyl or benzyl group; these compounds possibly being polyoxyalkylenated, notably polyoxyethylenated, and then preferably including from 1 to 50 ethylene oxide units, and more preferentially from 2 to 10 ethylene oxide units.

Preferably, the sulfate-type anionic surfactant(s) are selected from:
alkyl sulfates, notably C₈ to C₂₆, and preferably C₁₀ to C₂₂, alkyl sulfates;
alkyl ether sulfates, notably C₈ to C₂₆ and preferably C₁₀ to C₂₂ alkyl ether sulfates, preferably comprising from 1 to 10 ethylene oxide units;
in particular in the form of alkali metal, alkaline-earth metal, ammonium or amino alcohol salts; and
mixtures thereof.

When the anionic surfactant(s) of sulfate type are in salt form, said salt may be selected from alkali metal salts, such as the sodium or potassium salt, ammonium salts, amine salts and in particular amino alcohol salts, and alkaline earth metal salts, such as the magnesium salt, and mixtures thereof.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular sodium or magnesium salts are preferably used.

Preferably, the anionic surfactant(s) are selected from sodium, triethanolamine, magnesium or ammonium (C₁₀-C₂₂)alkyl sulfates, sodium, ammonium or magnesium (C₁₀-C₂₂)alkyl ether sulfates, which are oxyethylenated, for example with 1 or 2.2 mol of ethylene oxide, and mixtures thereof.

Even better still, the anionic surfactant(s) are selected from sodium, triethanolamine, ammonium or magnesium (C₁₀-C₂₂)alkyl sulfates, such as the compound sold under the name Texapon Z95P by the company BASF under the INCI name Sodium lauryl sulfate, and mixtures thereof.

Advantageously, the anionic surfactant(s) are selected from sodium lauryl sulfate, sodium laureth sulfate (SLES) and mixtures thereof.

According to another preferred embodiment, the anionic surfactant(s) are selected from:
acyl glutamates, notably C₆-C₃₀ or even C₈-C₂₆ acyl glutamates, such as stearoyl glutamates, and in particular disodium stearoyl glutamate;
acyl sarcosinates, notably C₆-C₃₀ or even C₈-C₂₆ acyl sarcosinates, such as palmitoyl sarcosinates, and in particular sodium palmitoyl sarcosinate;
(C₆-C₃₀)alkyl ether carboxylates, and notably (C₈-C₂₆)alkyl ether carboxylates;
and mixtures thereof.

The total amount of the anionic surfactant(s) present in the cosmetic composition of the invention is advantageously greater than or equal to 5% by weight, relative to the total weight of the composition. Preferably, the total amount of the anionic surfactant(s) present in the cosmetic composition of the invention ranges from 5% to 25% by weight, and more preferentially from 10% to 20% by weight, relative to the total weight of the composition.

In one particular embodiment of the invention, the cosmetic composition comprises one or more sulfate-type anionic surfactants, preferably selected from sodium, triethanolamine, ammonium or magnesium (C₁₀-C₂₂)alkyl sulfates and mixtures thereof. According to this embodiment, the total amount of the sulfate-type anionic surfactant(s) is advantageously greater than or equal to 5% by weight; this total amount preferably ranges from 5% to 25% by weight, and more preferentially from 10% to 20% by weight, relative to the total weight of the composition.

The weight ratio (R) of the total amount of the one or more anionic surfactants to the total amount of the one or more carboxylic acids of formula (I) as defined above that are present in the cosmetic composition of the invention is advantageously greater than or equal to 0.5 and preferably greater than or equal to 1. This weight ratio (R) more preferably ranges from 0.5 to 10 and better still from 1 to 5.

Preferably, the cosmetic composition according to the present invention is aqueous.

An “aqueous composition” according to the present invention means a composition in which the total water content is preferably greater than or equal to 20% by weight, relative to the weight of the composition. The water content is more preferably greater than or equal to 30% by weight, even more preferentially greater than or equal to 40% by weight of water, and even better still greater than or equal to 50% by weight, relative to the total weight of the composition. Advantageously, the total water content ranges from 20% to 85% by weight, relative to the total weight of the composition.

Besides water, the cosmetic composition according to the present invention may optionally comprise one or more organic solvents.

Preferably, the organic solvent(s) are chosen from linear or branched monoalcohols having from 1 to 8 carbon atoms and more preferentially from 1 to 4 carbon atoms, polyols, polyethylene glycols, aromatic alcohols and mixtures thereof.

As examples of organic solvents that can be used according to the invention, mention may notably be made of ethanol, propanol, butanol, isopropanol, isobutanol, propylene glycol, dipropylene glycol, isoprene glycol, butylene glycol, glycerol, sorbitol, benzyl alcohol and phenoxyethanol, and mixtures thereof.

The organic solvent(s) that may be used according to the invention may be chosen from linear or branched monoalcohols containing from 1 to 4 carbon atoms, and mixtures thereof, preferably from ethanol, propanol, butanol, isopropanol, isobutanol, and mixtures thereof.

The one or more organic solvents are preferably selected from polyols and mixtures thereof.

The pH of the cosmetic composition according to the invention varies preferably from 3 to 7, more preferably from 3.5 to 6 and better still from 4 to 5.

The pH of the cosmetic composition may be adjusted to the desired value by means of basifying agents or acidifying agents customarily used. Among the basifying agents, mention may be made, by way of examples, of aqueous ammonia, alkanolamines or mineral or organic hydroxides. Among the acidifying agents, mention may be made, by way of examples, of mineral or organic acids, such as hydrochloric acid, orthophosphoric acid, sulfuric acid, carboxylic acids other than the compounds (I) defined above, such as for example acetic acid, tartaric acid, citric acid or lactic acid, and sulfonic acids.

The cosmetic composition according to the present invention may optionally further comprise one or more additional compounds, different from the ingredients of the invention and among which mention may be made of fatty substances, cationic, anionic, nonionic, amphoteric or zwitterionic surfactants, cationic, anionic, nonionic or amphoteric polymers or mixtures thereof, anti-dandruff agents, anti-seborrhoea agents, agents for preventing hair loss and/or for promoting hair regrowth, vitamins and provitamins including panthenol, sunscreens, mineral or organic pigments, sequestrants, plasticizers, solubilizers, acidifying agents, mineral or organic thickeners, especially polymeric thickeners, opacifiers or nacreous agents, antioxidants, hydroxy acids, fragrances, preservatives, ceramides and mixtures thereof.

Of course, a person skilled in the art will take care to select this or these optional additional compounds such that the advantageous properties intrinsically attached to the cosmetic composition according to the invention are not, or not substantially, detrimentally affected by the envisaged addition(s).

The additional compounds above may generally be present in an amount, for each of them, of between 0% and 20% by weight, with respect to the total weight of the composition.

### The Cosmetic Treatment Method

The present invention also relates to a method for cosmetic treatment of keratin fibres, more particularly human keratin fibres such as the hair, comprising applying to said keratin fibres one or more carboxylic acids of formula (I), or optical isomers thereof, geometric isomers thereof, salts thereof, solvates thereof and mixtures thereof, as defined above.

The one or more carboxylic acids of formula (I) as defined above may advantageously be applied before, during and/or after a haircare or hair cleansing composition, such as a shampoo or a conditioner. The one or more carboxylic acids of formula (I) as defined above are preferably applied after a shampoo.

Another subject of the present invention relates to a method for cosmetic treatment of keratin fibres, more particularly human keratin fibres such as the hair, comprising applying to said keratin fibres a cosmetic composition as defined above.

Preferably, the methods for cosmetic treatment according to the present invention are methods for repairing and/or preventing the breakage of keratin fibres, more particularly human keratin fibres such as the hair.

The method according to the invention may be repeated two or more times, with or without a rest time between the various treatments, and/or with or without intermediate cosmetic treatment between two treatments.

The method according to the invention may therefore be implemented one or more times prior to a treatment such as bleaching and/or a durable shaping treatment such as a permanent-waving or a straightening treatment.

The one or more carboxylic acids of formula (I), optical isomers thereof, geometric isomers thereof, salts thereof, solvates thereof and mixtures thereof, or the cosmetic composition, as defined above, may be applied to dry or wet keratin fibres, preferably to wet keratin fibres.

The bath ratio of the cosmetic composition applied to the keratin fibres may range from 0.1 to 10. The term “bath ratio” means the ratio between the total weight of the cosmetic composition applied and the total weight of keratin fibres to be treated.

The methods for cosmetic treatment according to the present invention may comprise at least one additional successive step selected from steps ii) to iv) below:
ii) a step of placing the one or more carboxylic acids of formula (I), or optical isomers thereof, geometric isomers thereof, salts thereof, solvates thereof and mixtures thereof, or the cosmetic composition comprising them, on the keratin fibres, preferably for a time of at least 10 seconds, said placement step being carried out optionally under a hermetic film of curlpaper or plastic film type;
iii) a step of rinsing and/or washing the keratin fibers;
iv) a step of drying the keratin fibers in ambient air or using a heating device.

The placing step may have a duration ranging from 10 seconds to 60 minutes, preferably from 30 seconds to 30 minutes.

The washing step may be performed, for example, using a shampoo.

The temperature of the heating device may range from 45° C. to 230° C., preferably from 45° C. to 100° C., more preferentially from 50° C. to 80° C. A hairdryer, a heating hood, an iron or a heating brush may be used, for example, as heating device.

The drying step iv) may also be a combination of drying with air or with a heating device at a temperature ranging from 50° C. to 80° C., such as a hairdryer or a heating hood, followed by a step of treating with an iron.

The one or more carboxylic acids of formula (I), optical isomers thereof, geometric isomers thereof, salts thereof, solvates thereof and mixtures thereof, or the cosmetic composition, as defined above, may be integrated into a shampoo, a conditioner, a mask or any other formulation conventional in the haircare field.

The following examples serve to illustrate the invention without, however, having a limitative character.

### EXAMPLES

### I. Example 1—Application after Shampoo

a. Preparation of the formulations

Formulation A according to the invention and comparative formulation B were prepared from the ingredients stated in the table below.

TABLE 1

| | A (invention) | B (Comparative) |
|---|---|---|
| Tetrasodium N,N- | 421.71 mg | — |
| bis(carboxymethyl)-L-glutamate | | |
| HCl 1N | qs pH 4 | qs pH 4 |
| Water | qs 20 g | qs 20 g |

b. Protocol

Locks of Caucasian hair, damaged by two successive bleaching operations, weighing 2.7 g and 27 cm in length are washed with Elséve Color Vive shampoo and then wrung out between the fingers (2 passes). The locks are then placed on a flat surface and formulations A (invention) and B (comparative) are applied to said locks with a bath ratio of 0.4 (i.e. 0.4 g of formulation per 1 g of hair).

The hair is then massaged with the fingers, making circular movements along the entire length of each lock (5 times), and the locks are then left for 5 minutes. The locks are then rinsed with tap water for 30 seconds, wrung out and subsequently dried at 60° C. (10 min/g of hair). These various steps are repeated to give 5 applications in total.

The same protocol was reproduced on locks of damaged Chinese hair, using the same formulations A (invention) and B (comparative).

Locks of natural hair (Caucasian and Chinese), undamaged, were used as reference.
c. Study of hair fibre repair by differential scanning calorimetry

The differential scanning calorimetry technique is known to those skilled in the art as a method for quantifying the strengthening of proteins in the cortex of keratin fibres (Kinetics of the changes imparted to the main structural components of human hair by thermal treatment, https://doi.org/10.1016/j.tca.2018.01.014 & F.-J. Wortmann and H. Deutz, J. Appl. Polym Sci., 48, 137 (1993). The principle of the test is to measure the protein denaturing temperature. It is widely acknowledged that the higher the protein denaturing temperature, the better the integrity of the proteins of the cortex, which translates to a reduction in the breakage of the fibres.

The denaturing temperature is directly linked to the bonding density of the keratin proteins present in the cortex. Accordingly, the lower the denaturing temperature, the lower the bonding density between the proteins and the more the disulfide bridges break and the cortex is damaged. A difference of 2° C. is acknowledged by those skilled in the art as a significant modification.

The instrument used for performing the measurements is a TA Instruments DSC2500 reference instrument (temperature range: 30° C.-200° C.; heating ramp of 5° C./min). This instrument measures the energy flow during heating of the sample. The temperature of maximum energy flow represents the denaturing temperature.

The results of the denaturing temperature measurements for each of the locks treated according to the protocol described above are summarized in the table below and correspond to the mean of the measurements carried out on 6 hair strands.

TABLE 2

| | | Denaturing |
|---|---|---|
| | | temperature (° C.), |
| | | mean for 6 hair |
| Type of hair | Treatment of the lock | samples |
| Caucasian | Lock treated with formulation A - | 158 |
| | invention | |
| | Lock treated with formulation B - | 141 |
| | comparative | |
| | Natural lock, neither damaged nor | 151 |
| | treated - reference | |
| Chinese | Lock treated with formulation A - | 154 |
| | invention | |
| | Lock treated with formulation B - | 144 |
| | comparative | |
| | Natural lock, neither damaged nor | 153 |
| | treated - reference | |

The results above show that the use of the carboxylic acids of formula (I) as defined above increases the bonding density of the keratin proteins present in the cortex of the treated hair, so enabling repair of the damaged hair.

The reason is that the denaturing temperatures measured on the locks treated according to the present invention (formulation A) are higher, irrespective of hair type (Caucasian or Chinese), than the denaturing temperatures measured for the locks treated with the comparative formulation B.

Furthermore, the denaturing temperature for the locks treated according to the present invention is equivalent to (Chinese hair) or even better than (Caucasian hair) that measured for the natural and undamaged hair, thus demonstrating that the hair is repaired.
d. Study of hair breakage via the cyclic fatigue test

The cyclic fatigue technique is known to those skilled in the art for the breakage of keratin fibres (reference: https://www.diastron.com/app/uploads/2017/06/Dia-Stron-CYC801-Brochure.pdf/). The principle of the test is to measure the number of cycles of extension of a single fibre before breakage. Fibres 30 mm in length are subjected to a constant stress (130 MPa) at a constant rate (40 mm/s). The studies are carried out at 25° C. and 45% relative humidity. A Diastron Cyclic instrument is used.

The number of cycles measured for each of the locks treated according to the protocol described above are summarized in the table below and correspond to the mean of the measurements carried out on 50 hair strands.

TABLE 3

| | | Number of cycles |
|---|---|---|
| | | before breakage |
| | | (mean for 50 |
| Type of hair | Treatment of the lock | strands) |
| Chinese | Lock treated with formulation A - | 13,539 |
| | invention | |
| | Lock treated with formulation B - | 8,869 |
| | comparative | |

The results above show that the use of the carboxylic acids of formula (I) as defined above improves the strength of the keratin fibres and so prevents them breaking.

The reason is that the locks treated according to the present invention (formulation A) break only after 13 539 cycles, whereas the locks treated with the comparative formulation B break at 8 869 cycles.

### II. Example 2

a. Preparation of the compositions

The compositions A1, A2 and A3 according to the invention were prepared from the ingredients stated in the table below (% in g of active substance).

TABLE 4

| | A1 | A2 | A3 |
|---|---|---|---|
| Tetrasodium N,N- | 5 | 10 | 15 |
| bis(carboxymethyl)-L-glutamate | | | |
| Sodium laureth sulfate | 15 | 15 | 15 |
| HCl 1N | qs pH 4 | qs pH 4 | qs pH 4 |
| Water | qs 100 | qs 100 | qs 100 |

b. Protocol

Locks of damaged Caucasian hair, weighing 2.7 g and 27 cm in length, are washed with compositions A1, A2 and A3 described above, using 0.4 g of composition per 1 g of hair. The locks are massaged with the fingers, making circular movements all along each lock (5 times), then the locks are left for 1 minute on a flat, non-absorbent surface (such as a sink unit edge). The locks are subsequently rinsed with tap water for 30 seconds and then wrung out.

This same protocol is repeated a second time for each lock. At the end of the “second” wringing, the locks are dried at 60° C. (10 min/g of hair).

These various steps (drying included) are repeated to give 5 applications in total.

The same protocol was reproduced on locks of damaged Chinese hair, using the same compositions A1, A2 and A3.

The commercial shampoo composition DOP was used as a comparative on locks of damaged hair (Caucasian and Chinese), according to the same protocol.

Locks of natural hair (Caucasian and Chinese), undamaged, were used as reference.
c. Study of hair fibre repair by differential scanning calorimetry

The differential scanning calorimetry technique is known to those skilled in the art as a method for quantifying the strengthening of proteins in the cortex of keratin fibres (Kinetics of the changes imparted to the main structural components of human hair by thermal treatment, https://doi.org/10.1016/j.tca.2018.01.014 & F.-J. Wortmann and H. Deutz, J. Appl. Polym Sci., 48, 137 (1993). The principle of the test is to measure the protein denaturing temperature. It is widely acknowledged that the higher the protein denaturing temperature, the better the integrity of the proteins of the cortex, which translates to a reduction in the breakage of the fibres.

The denaturing temperature is directly linked to the bonding density of the keratin proteins present in the cortex. Accordingly, the lower the denaturing temperature, the lower the bonding density between the proteins and the more the disulfide bridges break and the cortex is damaged. A difference of 2° C. is acknowledged by those skilled in the art as a significant modification.

The instrument used for performing the measurements is a TA Instruments DSC2500 reference instrument (temperature range: 30° C.-200° C.; heating ramp of 5° C./min). This instrument measures the energy flow during heating of the sample. The temperature of maximum energy flow represents the denaturing temperature.

The results of the denaturing temperature measurements for each of the Locks according to the protocol described above are summarized in the table below and correspond to the mean of the measurements carried out on 6 hair strands.

TABLE 5

| | | Denaturing |
|---|---|---|
| | | temperature (° C.), |
| | | mean for 6 hair |
| Type of hair | Treatment of the lock | strands |
| Caucasian | Lock washed with composition | 144 |
| | A1 - invention | |
| | Lock washed with composition | 148 |
| | A2 - invention | |
| | Lock washed with composition | 150 |
| | A3 - invention | |
| | Lock washed with commercial | 139 |
| | shampoo (DOP) - reference | |
| | Natural lock, neither damaged | 151 |
| | nor treated - reference | |
| Chinese | Lock washed with composition | 147 |
| | A1 - invention | |
| | Lock washed with composition | 148 |
| | A2- invention | |
| | Lock washed with composition | 149 |
| | A3- invention | |
| | Lock washed with commmercial | 145 |
| | shampoo (DOP) - reference | |
| | Natural lock, neither damaged | 153 |
| | nor treated - reference | |

The results above show that the composition of the invention, comprising at least one carboxylic acid of formula (I) as defined above, increases the bonding density of the keratin proteins present in the cortex of the treated hair, so enabling prevention of breakage, or even repair, of the damaged hair.

The reason is that the denaturing temperatures measured on the locks treated with the compositions of the invention (A1, A2 and A3) are higher, irrespective of hair type (Caucasian or Chinese), than the denaturing temperatures measured on the locks treated with the commercial shampoo composition.

Furthermore, the denaturing temperatures measured after application of the compositions according to the invention (A1, A2 and A3) are also closer to the denaturing temperature measured on the natural and undamaged hair, thereby demonstrating that the composition of the invention enables the damaged hair to be repaired, irrespective of its type (Caucasian or Chinese).
d. Study of hair breakage via the flexabrasion test

The principle of the flexabrasion test is to measure the strength of a dry hair subjected to repeated stresses of extension and abrasion (Cosmetics & Toiletries, Flexabrasion: A Method for Evaluating Hair Strength, Jun. 26, 2009). A strand of hair is attached to a 20 g weight at one end and the other end is attached to an immobile bar. The strand of hair moves back and forth over a 300 m stainless steel wire. The movement has an amplitude of 10 mm and a frequency of 0.5 Hz.

Breakage is detected by an optical sensor which records the lifetime of the hair (also called “drop time before breakage”, i.e. the time that the hair takes to break). The longer this time, the more breakage-resistant the hair.

One series of measurements comprises 75 hair strands. A difference in drop time of 100 s is acknowledged by those skilled in the art as a significant modification.

The results of the drop time before breakage measurements for each of the locks treated according to the protocol described above are summarized in the table below and correspond to the mean of the measurements carried out on 75 hair strands.

TABLE 6

| | | Drop time |
|---|---|---|
| | | before breakage |
| Type of | | (median - in |
| hair | Treatment of the lock | seconds) |
| Caucasian | Lock washed with composition A1 - | 883 |
| | invention | |
| | Lock washed with composition A2 - | 730 |
| | invention | |
| | Lock washed with composition A3 - | 709 |
| | invention | |
| | Lock washed with commmercial | 549 |
| | shampoo (DOP) - reference | |
| | Natural lock, neither damaged | 794 |
| | nor treated - reference | |
| Chinese | Lock washed with composition A1 - | 2 022 |
| | invention | |
| | Lock washed with composition A2 - | |
| | invention | |
| | Lock washed with composition A3 - | 2 020 |
| | invention | |
| | Lock washed with commmercial | 1 435 |
| | shampoo (DOP) - reference | |
| | Natural lock, neither damaged | 1 780 |
| | nor treated - reference | |

The results above show that the hair locks treated with the compositions of the invention (A1, A2 and A3) are more resistant to breakage than the locks treated with the commercial shampoo composition. Indeed, regardless of the type of hair (Caucasian or Chinese), after application of the compositions of the invention, the locks take longer to break than those treated with the commercial composition.

Furthermore, the drop time for the locks treated with the compositions of the invention is equivalent to (Caucasian hair) or better than (Chinese hair) the drop time recorded for the natural and undamaged hair, thus demonstrating that the hair is repaired.

1. Use of one or more carboxylic acids of formula (I) below, or optical isomers thereof, geometric isomers thereof, salts thereof, solvates thereof and mixtures thereof:
R₁—N—(CH(R₂)COOH)₂ (I)
in which:
R₁ represents a hydrogen atom or a —CH(COOH)—(CH₂)₂—COOH, —CH₂CH₂OH, —CH(CH₃)COOH, —(CH₂)₂N(COR₃)—CH₂—COOH or —CH(COOH)—CH₂—COOH group; and
R₂ represents a CH₂COOH group when R₁ represents a hydrogen atom or R₂ represents a hydrogen atom when R₁ is other than a hydrogen atom; and
R₃ represents a linear or branched alkyl group comprising from 1 to 4 carbon atoms or cyclic alkyl group comprising from 3 to 30 carbon atoms,
for repairing and/or preventing the breakage of keratin fibres, more particularly human keratin fibres such as the hair.

2. Use according to claim 1, characterized in that the keratin fibres are damaged and/or sensitized keratin fibres.

3. Use according to claim 1, characterized in that the one or more carboxylic acids of formula (I) are selected from methylglycinediacetic acid, N-lauroylethylenediamine-N,N′,N′-triacetic acid, N,N-dicarboxymethylglutamic acid, iminodisuccinic acid, N,N-bis(carboxymethyl)aspartic acid, alkali metal salts thereof, alkaline earth metal salts thereof, transition metal salts thereof, organic amine salts thereof, ammonium salts thereof, optical isomers thereof, geometric isomers thereof, solvates thereof and mixtures thereof; and preferably from N,N-dicarboxymethylglutamic acid, N,N-bis(carboxymethyl)aspartic acid, alkali metal salts thereof, alkaline earth metal salts thereof, transition metal salts thereof, organic amine salts thereof, ammonium salts thereof, optical isomers thereof, geometric isomers thereof, solvates thereof and mixtures thereof.

4. Use according to claim 1,
characterized in that the one or more carboxylic acids of formula (I) are selected from N,N-dicarboxymethyl-L-glutamic acid, N,N-bis(carboxymethyl)-L-aspartic acid, alkali metal salts thereof, alkaline earth metal salts thereof, transition metal salts thereof, organic amine salts thereof, ammonium salts thereof, optical isomers thereof, geometric isomers thereof, solvates thereof and mixtures thereof; and preferably the carboxylic acid of formula (I) is N,N-dicarboxymethyl-L-glutamic acid and/or tetrasodium N,N-bis(carboxymethyl)-L-glutamate.

5. Cosmetic composition comprising:
(i) from 2% to 25% by weight, relative to the total weight of the composition, of one or more carboxylic acids of formula (I) below, or optical isomers thereof, geometric isomers thereof, salts thereof, solvates thereof and mixtures thereof:
R₁—N—(CH(R₂)COOH)₂ (I)
in which:
R₁ represents a hydrogen atom or a —CH(COOH)—(CH₂)₂—COOH, —CH₂CH₂OH, —CH(CH₃)COOH, —(CH₂)₂N(COR₃)—CH₂—COOH or —CH(COOH)—CH₂—COOH group; and
R₂ represents a CH₂COOH group when R₁ represents a hydrogen atom or R₂ represents a hydrogen atom when R₁ is other than a hydrogen atom; and
R₃ represents a linear or branched alkyl group comprising from 1 to 4 carbon atoms or cyclic alkyl group comprising from 3 to 30 carbon atoms, and
(ii) one or more anionic surfactants.

6. Composition according to claim 5, characterized in that the one or more carboxylic acids of formula (I) are selected from methylglycinediacetic acid, N-lauroylethylenediamine-N,N′,N′-triacetic acid, N,N-dicarboxymethylglutamic acid, iminodisuccinic acid, N,N-bis(carboxymethyl)aspartic acid, alkali metal salts thereof, alkaline earth metal salts thereof, transition metal salts thereof, organic amine salts thereof, ammonium salts thereof, optical isomers thereof, geometric isomers thereof, solvates thereof and mixtures thereof; and preferably from N,N-dicarboxymethylglutamic acid, N,N-bis(carboxymethyl)aspartic acid, alkali metal salts thereof, alkaline earth metal salts thereof, transition metal salts thereof, organic amine salts thereof, ammonium salts thereof, optical isomers thereof, geometric isomers thereof, solvates thereof and mixtures thereof.

7. Composition according to claim 5, characterized in that the one or more carboxylic acids of formula (I) are selected from N,N-dicarboxymethyl-L-glutamic acid, N,N-bis(carboxymethyl)-L-aspartic acid, alkali metal salts thereof, alkaline earth metal salts thereof, transition metal salts thereof, organic amine salts thereof, ammonium salts thereof, optical isomers thereof, geometric isomers thereof, solvates thereof and mixtures thereof; and preferably the carboxylic acid of formula (I) is N,N-dicarboxymethyl-L-glutamic acid and/or tetrasodium N,N-bis(carboxymethyl)-L-glutamate.

8. Composition according to claim 5, characterized in that the total amount of the one or more carboxylic acids of formula (I) ranges from 2% to 20% by weight, relative to the total weight of the composition.

9. Composition according to claim 5, characterized in that the one or more anionic surfactants are selected from:
alkyl sulfates, in particular C₈ to C₂₆, and preferably C₁₀ to C₂₂, alkyl sulfates;
alkyl ether sulfates, in particular C₈ to C₂₆ and preferably C₁₀ to C₂₂ alkyl ether sulfates, more preferentially comprising from 2 to 10 ethylene oxide units;
more particularly in the form of alkali metal, alkaline earth metal, ammonium or amino alcohol salts, and
mixtures thereof.

10. Composition according to claim 5, characterized in that the one or more anionic surfactants are selected from sodium, triethanolamine, magnesium or ammonium alkyl(C₁₀-C₂₂) sulfates, ethoxylated sodium, ammonium or magnesium alkyl(C₁₀-C₂₂) ether sulfates, and mixtures thereof; preferably from sodium, triethanolamine, ammonium or magnesium alkyl(C₁₀-C₂₂) sulfates and mixtures thereof; and more preferably from sodium lauryl sulfate, sodium laureth sulfate and mixtures thereof.

11. Composition according to claim 5, characterized in that the total amount of the one or more anionic surfactants is greater than or equal to 5% by weight, preferably from 5% to 25% by weight and more preferentially from 10% to 20% by weight, relative to the total weight of the composition.

12. Composition according to claim 5, characterized in that the weight ratio of the total amount of the one or more anionic surfactants to the total amount of the one or more carboxylic acids of formula (I) ranges from 0.5 to 10 and preferably from 1 to 5.

13. Composition according to claim 5, characterized in that the pH of said composition varies from 3 to 7, preferably from 3.5 to 6 and more preferentially from 4 to 5.

14. Composition according to claim 5, characterized in that it further comprises water; preferably in a total amount greater than or equal to 20% by weight; more preferentially greater than or equal to 30% by weight; better still greater than or equal to 40% by weight of water; and even better greater than or equal to 50% by weight, relative to the total weight of the composition.

15. Method for cosmetic treatment of keratin fibres, more particularly human keratin fibres such as the hair, comprising applying to said keratin fibres a cosmetic composition as defined in claim 5.
